(19) United States
(12) Patent Application Publication
(10) Pub. No.: US 2023/0390063 A1
(43) Pub. Date: Dec. 7, 2023

(54) SYSTEMS, APPARATUSES, AND METHODS FOR PAPILLARY MUSCLE APPROXIMATION

(71) Applicant: EDWARDS LIFESCIENCES CORPORATION, Irvine, CA (US)

(72) Inventor: Brian S. Conklin, Orange, CA (US)

(21) Appl. No.: 18/454,237

(22) Filed: Aug. 23, 2023

Related U.S. Application Data
(63) Continuation of application No. PCT/US2022/017219, filed on Feb. 22, 2022.
(60) Provisional application No. 63/152,670, filed on Feb. 23, 2021.

(51) Int. Cl. A61F 2/24

(52) U.S. Cl. CPC A61F 2/2487; A61F 2002/249; A61F 2220/0008; A61F 2220/0075

(57) ABSTRACT

Systems, apparatuses, and methods disclosed herein are provided for medical treatment, including treatment of dilated hearts (e.g., dilated left ventricle) or functional mitral valve regurgitation within a human heart. In instances, transcatheter medical treatments may be utilized. The portion of the patient's heart may be dilated due to a myocardial infarction or other cardiomyopathy. The treatment may comprise beating-heart repair of left ventricles with ischemic or non-ischemic dilated cardiomyopathy. The treatments may include approximating papillary muscles of the heart.

### CROSS-REFERENCE TO RELATED APPLICATIONS

This application is a continuation of International Application No. PCT/US2022/017219, filed Feb. 22, 2022, which claims the benefit of U.S. Patent Application No. 63/152,670, filed Feb. 23, 2021, the entire disclosures all of which are incorporated by reference for all purposes.

### BACKGROUND

Heart failure can occur when the left ventricle of the heart becomes enlarged and dilated as a result of one or more of various etiologies. Initial causes of heart failure can include chronic hypertension, myocardial infarction, mitral valve incompetency, and other dilated cardiomyopathies. With each of these conditions, the heart is forced to overexert itself in order to provide a cardiac output demanded by the body during various demand states. The result can be an enlarged left ventricle.

A dilated or enlarged heart, and particularly a dilated or enlarged left ventricle, can significantly increase tension and stress in heart walls both during diastolic filling and systolic contraction, which contributes to further dilatation or enlargement of chambers of the heart. In addition, mitral valve incompetency or mitral valve regurgitation is a common comorbidity of congestive heart failure. As the dilation of the ventricle increases, valve function generally worsens, which results in a volume overload condition. The volume overload condition further increases ventricular wall stress, thereby advancing the dilation process, which further worsens valve dysfunction.

In heart failure, the size of the valve annulus (particularly the mitral valve annulus) increases while the area of the leaflets of the valve remains constant. This may lead to reduced coaptation area between the valve leaflets, and, as a result, eventually to valve leakage or regurgitation. Moreover, in normal hearts, the annular size contracts during systole, aiding in valve coaptation. In heart failure, there is poor ventricular function and elevated wall stress. These conditions tend to reduce annular contraction and distort annular size, often exacerbating mitral valve regurgitation. In addition, as the chamber dilates, the papillary muscles (to which the leaflets are connected via the chordae tendineae) may move radially outward and downward relative to the valve, and relative to their normal positions. During this movement of the papillary muscles, however, the various chordae lengths remain substantially constant, which limits the full closure ability of the leaflets by exerting tension prematurely on the leaflets. This condition is commonly referred to as “chordal tethering.” The combination of annular changes and papillary changes results in a poorly functioning valve.

### SUMMARY

Systems, apparatuses, and methods disclosed herein are provided for medical treatment, including treatment of dilated hearts (e.g., dilated left ventricle) or functional mitral valve regurgitation within a human heart. In some examples, transcatheter medical treatments may be utilized. The portion of the patient's heart may be dilated due to a myocardial infarction or other cardiomyopathy. The treatment may comprise beating-heart repair of left ventricles with ischemic or non-ischemic dilated cardiomyopathy. The treatments may include approximating papillary muscles of the heart.

The systems, apparatuses, and methods disclosed herein may include applying one or more heart splints to the patient's heart to apply pressure to the heart to approximate the papillary muscles. The heart splints may include anchors connected by a tension member that is tensioned to apply pressure to the patient's heart. The anchors may be positioned in desired locations to approximate the papillary muscles and reshape the heart at particular locations.

In certain examples, the systems, apparatuses, and methods disclosed herein may be utilized in a minimally invasive procedure, to access the heart and apply the heart splint without requiring a full sternotomy.

Any or all of the treatment methods, operations, or steps described herein may be performed as simulations on a living human or non-human subject, or on a human or non-human cadaver or portion(s) thereof (e.g., heart, body part, tissue, etc.), simulator, or anthropomorphic ghost, for example, for educational, or training purposes.

A heart anchor of the present disclosure may include a ring having two ends and configured to move from a linearized configuration to a ring-shaped configuration, a first portion of the ring overlapping a second portion of the ring in the ring-shaped configuration. The heart anchor may include a cover coupled to the ring and extending inward from the ring in the ring-shaped configuration.

A heart anchor of the present disclosure may be for a heart splint, and may be for a system disclosed herein. The system may be for approximating papillary muscles of a heart. The system may include a first heart anchor configured to be positioned on an external posterior surface of the heart proximate a papillary muscle of the heart. The system may include a second heart anchor configured to be positioned on an external anterior surface of the heart proximate a papillary muscle of the heart. The system may include a tension member configured to couple the first heart anchor to the second heart anchor and extend within a ventricle of the heart.

A method as disclosed herein may include approximating papillary muscles of a heart. The method may include deploying a first heart anchor to an external posterior surface of the heart proximate a papillary muscle of the heart. The method may include deploying a second heart anchor to an external anterior surface of the heart proximate a papillary muscle of the heart. The method may include tensioning a tension member for coupling the first heart anchor to the second heart anchor. The method may include locking the tension member in tension between the first heart anchor and the second heart anchor across a ventricle.

### BRIEF DESCRIPTION OF THE DRAWINGS

Features and advantages of the systems, apparatuses, and methods as disclosed herein will become appreciated as the same become better understood with reference to the specification, claims, and appended drawings wherein:

FIG. 1A illustrates a cross sectional view of a portion of a heart.

FIG. 1B illustrates a cross sectional view of a portion of a dilated heart suffering from functional mitral valve regurgitation.

FIG. 2A illustrates a front view of a ring of a heart anchor according to an example of the present disclosure.

FIG. 2B illustrates a side view of the ring of a heart anchor shown in FIG. 2A.

FIG. 2C illustrates a top view of an unfolded cover of a heart anchor according to an example of the present disclosure.

FIG. 2D illustrates a top view of the cover shown in FIG. 2C folded.

FIG. 2E illustrates a side view of the folded cover shown in FIG. 2D.

FIG. 2F illustrates an alternate configuration of a cover of a heart anchor according to an example of the present disclosure.

FIG. 2G illustrates a heart anchor according to an example of the present disclosure.

FIG. 2H illustrates the heart anchor shown in FIG. 2G in a linearized configuration.

FIG. 3A illustrates a side view of a deployment apparatus with an anchor partially extending out of a deployment apparatus according to an example of the present disclosure.

FIG. 3B illustrates a side view of a deployment apparatus with an anchor extending out of a deployment apparatus according to an example of the present disclosure.

FIG. 4A illustrates a cross sectional view of a patient's heart with a deployment apparatus passing through the patient's heart according to an example of the present disclosure, and a heart anchor being deployed.

FIG. 4B illustrates a cross sectional view of a patient's heart with a deployment apparatus passing through the patient's heart according to an example of the present disclosure, and a heart anchor being deployed.

FIG. 4C illustrates a cross sectional view of a patient's heart with a heart splint being deployed to the patient's heart according to an example of the present disclosure.

FIG. 4D illustrates a cross sectional view of a patient's heart with a heart splint deployed to the patient's heart according to an example of the present disclosure.

FIG. 4E illustrates a top cross sectional view of a patient's heart with a heart splint deployed to the patient's heart according to an example of the present disclosure.

FIG. 5A illustrates a cross sectional view of a heart anchor according to an example of the present disclosure.

FIG. 5B illustrates a cross sectional view of the heart anchor shown in FIG. 5A according to an example of the present disclosure.

FIG. 6A illustrates a cross sectional view of a patient's heart with a puncturing device passing through the left ventricle according to an example of the present disclosure.

FIG. 6B illustrates a cross sectional view of a patient's heart with a puncturing device passing through the left ventricle according to an example of the present disclosure.

FIG. 6C illustrates a cross sectional view of a patient's heart with a tension member being drawn through the left ventricle according to an example of the present disclosure.

FIG. 6D illustrates a cross sectional view of a patient's heart with a heart splint deployed to the patient's heart according to an example of the present disclosure.

FIG. 7 illustrates a cross sectional view of a patient's heart with a tension member being drawn through the left ventricle according to an example of the present disclosure.

FIG. 8A illustrates a cross sectional view of a patient's heart with a heart anchor being deployed to a posterior wall of the heart according to an example of the present disclosure.

FIG. 8B illustrates a cross sectional view of a patient's heart with a heart anchor being deployed to an anterior wall of the heart according to an example of the present disclosure.

FIG. 8C illustrates a cross sectional view of a patient's heart with a heart splint deployed to the patient's heart according to an example of the present disclosure.

### DETAILED DESCRIPTION

Various aspects of the present disclosure generally relate to systems, apparatuses, and methods for medical treatment and/or treating heart conditions, including, by way of example, treating dilation/dilatation (including a dilated left ventricle), valve incompetencies (including mitral valve regurgitation), and other similar heart conditions. The systems, apparatuses, and methods in some examples may be adapted for transcatheter medical treatments that may not require full, open surgery, and can be minimally invasive. The systems, apparatus, and methods may be utilized to approximate one or more papillary muscles of a patient's heart, and may reshape the left ventricle in some examples.

In certain examples, the present disclosure involves geometric reshaping of the heart and treating valve incompetencies. In certain aspects of the present disclosure, the papillary muscles may be approximated to reduce chordal tethering, to relieve functional mitral regurgitation. In some examples, the systems, apparatuses, and methods may be utilized to approximate papillary muscles of the right ventricle, to relive functional tricuspid regurgitation.

In some examples, the systems, apparatuses, and methods disclosed herein may be utilized in a beating heart procedure. Such a procedure may involve deployment of the heart splint while the heart is beating. In such an example, the papillary muscle approximation amount may be fine tuned while monitoring hemodynamics in real-time and locking the heart splint in position when a desired result is reached. Rapid pacing may be utilized at desired times to minimize heart motion and improve ease of targeting desired puncture locations on the heart.

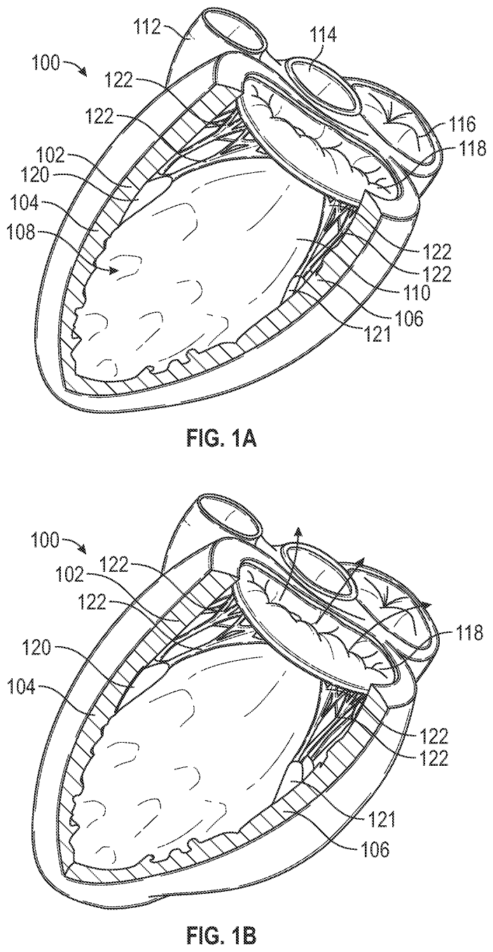

FIG. 1A illustrates an example of a heart 100, with a partial cross sectional view of the left ventricle 102. An anterior wall 104 of the left ventricle 102 is shown as well as a posterior wall 106 of the left ventricle 102. The interior cavity 108 of the left ventricle 102, and the interventricular septum no are shown. The pulmonary artery 112, aorta 114, and tricuspid valve 116 are also shown in a representative view.

The mitral valve 118 is shown in FIG. 1A. The heart 100 shown in FIG. 1A represents a normally operating heart, in which the leaflets of the mitral valve 118 coapt properly and the annulus of the mitral valve 118 has a shape allowing the leaflets of the mitral valve 118 to coapt properly. The papillary muscles 120, 121 are positioned relative to each other and relative to the mitral valve 118 such that the chordae 122 extending to the mitral valve leaflets properly tether to the leaflets and allow the leaflets to close and open in a desired manner.

The heart wo may suffer from maladies that alter the position of the papillary muscles 120, 121. Such maladies may comprise enlargement of the left ventricle 102. Such enlargement may be caused by ischemic or non-ischemic dilated cardiomyopathy, among other maladies. Abnormal tethering forces on the chordae 122 may result from displacement of the papillary muscles 120, 121 due to enlargement of the left ventricle 102.

FIG. 1B, for example, illustrates the heart wo with a dilated left ventricle 102. The papillary muscles 120, 121 have moved away from each other thus producing abnormal tethering forces upon the chordae 122. The leaflets of the mitral valve 118 may not coapt properly, thus leading to functional mitral regurgitation as represented by the arrows in FIG. 1B.

Treatment for such a condition may comprise approximating the papillary muscles 120, 121 to reduce the tension upon the chordae 122 and thus allowing the leaflets to return closer to a native state of coaptation. A heart splint as disclosed herein may be utilized to approximate the papillary muscles 120, 121. The heart splint may include heart anchors and a tension member configured to couple the heart anchors to each other and extend within a ventricle of the heart.

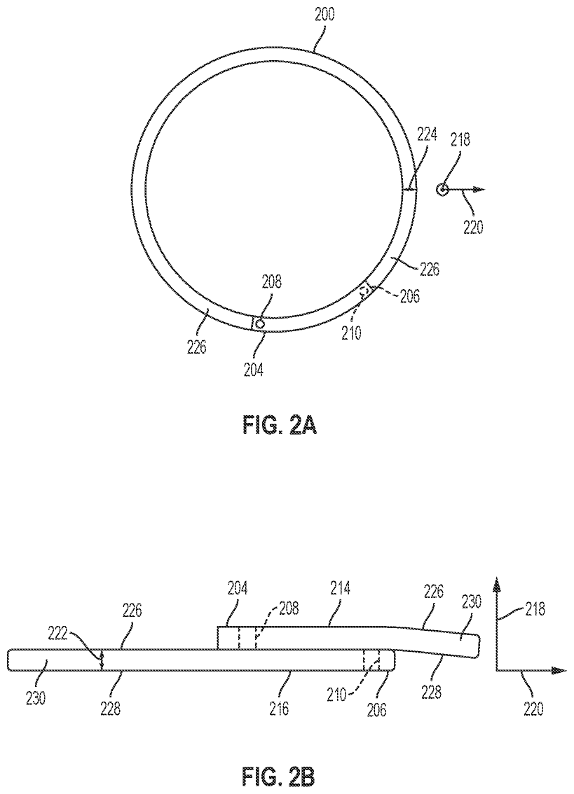

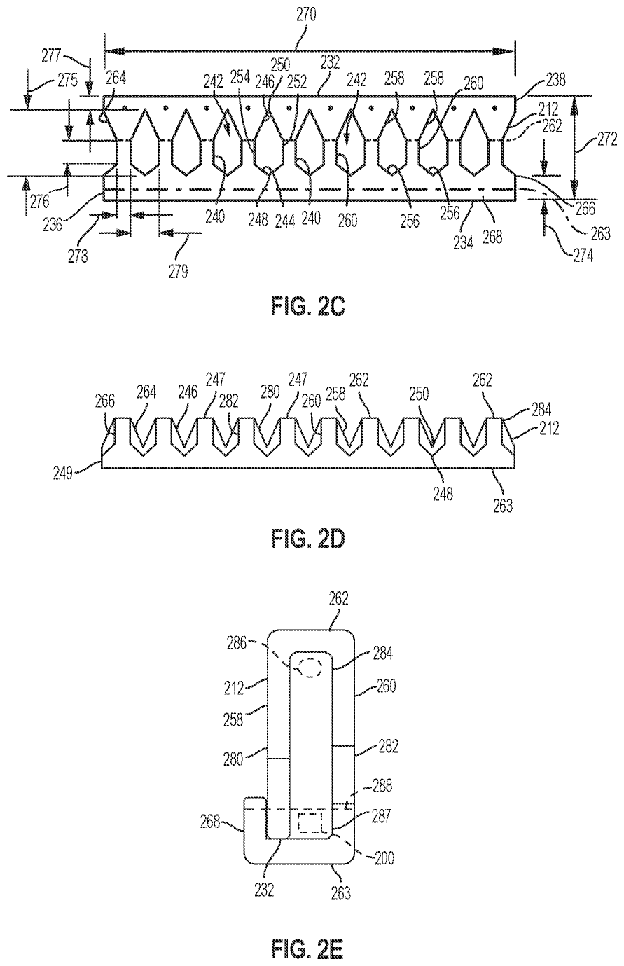

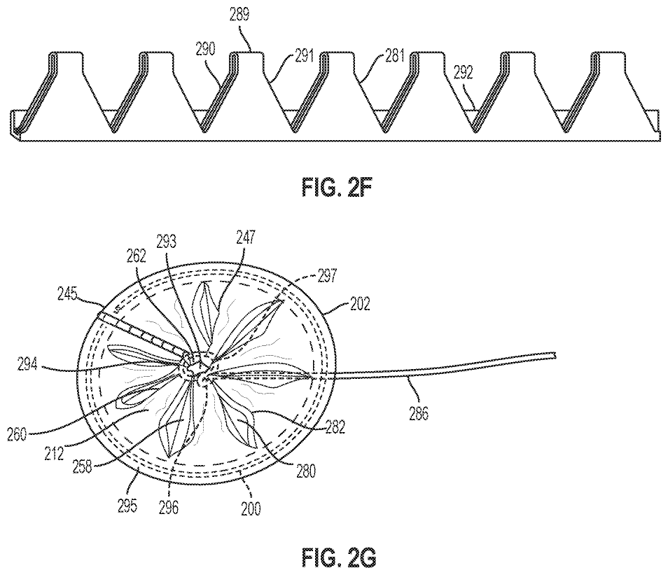

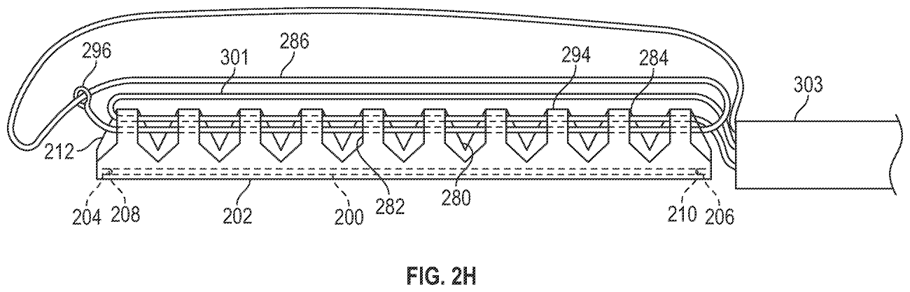

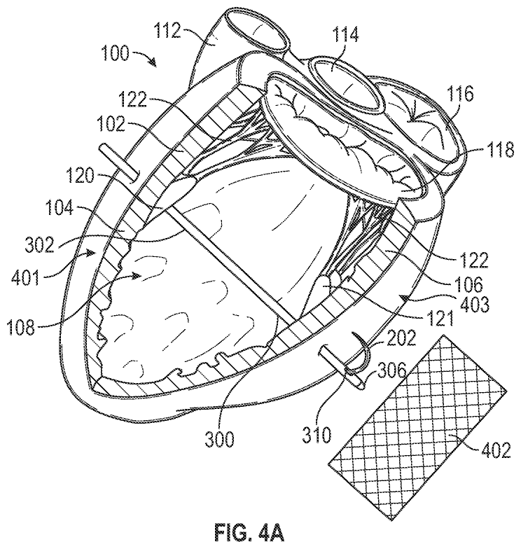

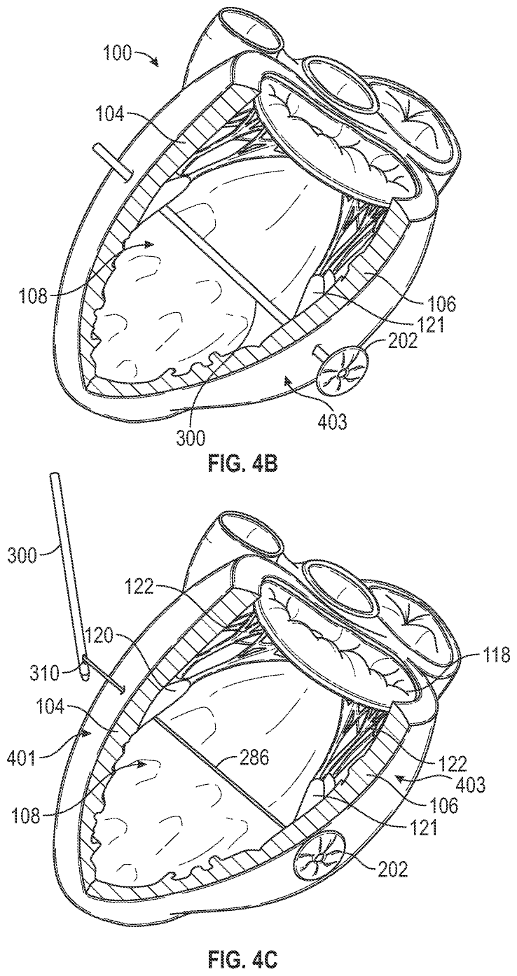

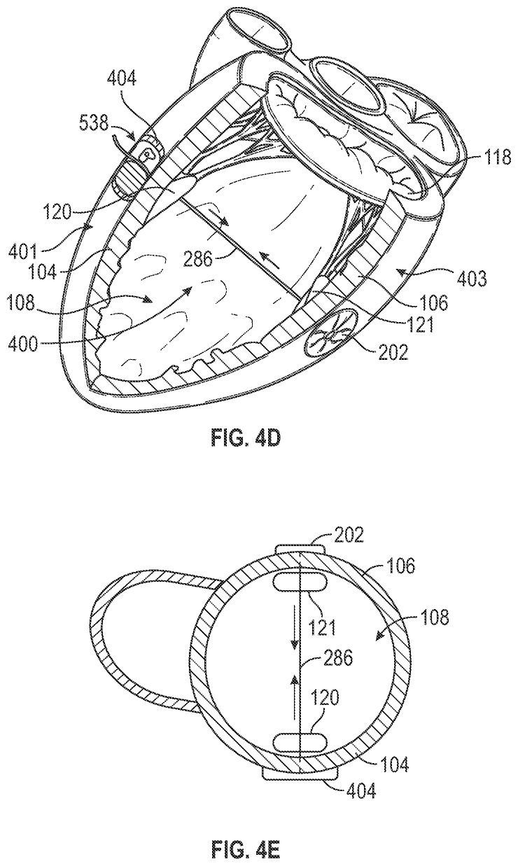

FIG. 2A illustrates a component of a heart anchor that may be utilized in some examples herein. Features of heart anchors may be disclosed in U.S. patent application Ser. No. 16/549,957, titled METHODS AND DEVICES FOR VENTRICULAR RESHAPING AND HEART VALVE RESHAPING, filed on Aug. 23, 2019 and published as U.S. Publication No. 2020/0069426, the entire contents of which are incorporated herein for all purposes. FIG. 2A illustrates an example of a ring 200 that may be used in a heart anchor, as may be used in the systems and methods disclosed herein. A heart anchor 202 as may be used in the systems and methods disclosed herein is illustrated in FIG. 2G. The heart anchor 202 may be utilized in a heart splint (for example the splint 400 shown in FIG. 4D, or other splints).

The ring 200 may include a body having a first end 204 and a second end 206 (shown in FIG. 2A in dashed lines, and shown in FIG. 2B). The ring 200 may be configured to move from a linearized configuration (as shown in FIG. 2H) to a ring-shaped configuration, as shown in FIG. 2A. The first end 204 may include an opening 208 extending through the ring 200 at or proximal the first end 204 and an opening 210 (shown in FIGS. 2A and 2B in dashed lines) extending through the ring 200 at or proximal the second end 206. The openings 208, 210 may comprise couplers for coupling to the cover 212 (shown in FIGS. 2G and 2H).

FIG. 2B illustrates a side view of the ring 200 in the ring-shaped configuration shown in FIG. 2A. Portions 214, 216 of the ring 200 may overlap when the ring 200 is in the ring-shaped configuration. The portions 214, 216 may overlap in the axial dimension 218, as opposed to the radial dimension 220. The portions 214, 216 may overlap such that the portions 214, 216 that overlap include the ends 204, 206. From a top view (as shown in FIG. 2A), the portions may overlap such that the edges of the body of the ring 200 have a matching profile as viewed from the top. The edges of the body of the ring 200 may be aligned with each other in the radial dimension 220. The edges of the body of the ring are not offset from each other at the overlapping portions in the radial dimension 220. Thus, the ring 200 may appear as a continuous ring, which may have a circular shape or other shape as desired. Some examples of the ring include any suitable closed shape, which can be generally flat, as illustrated in FIGS. 2A and 2B, or can have a three-dimensional shape, for example, that accommodates one or more anatomical features.

In some examples, a first end portion of the ring can overlap a second end portion of the ring where at least one of the first end portion or the second end portion is adjacent to or spaced from the respective end. For example, in some rings, at least one edge of the first end portion is offset or at an angle to at least one edge of the second end portion at the overlap. In other examples, the overlapping portions can include both ends of the first and second end portions where at least one edge of the first end portion is offset from an edge of the second end portion.

The overlapping portions 214, 216 may contact each other, and one of the overlapping portions may provide a support against force for the other overlapping portion. For example, a force applied to portion 214 may be resisted by portion 216 at the overlap, and a force applied to portion 216 may be resisted by portion 214 at the overlap. The overlapping portions 214, 216 may provide support for the ring 200 upon a force being applied in the axial dimension.

The overlapping portions 214, 216 may overlap to a desired amount. In one example, the overlapping portions 214, 216 may overlap to at least about 5 degrees of the ring 200. In one example, the overlapping portions 214, 216 may overlap to at least about 10 degrees, to at least about 20 degrees, to at least about 40 degrees, or to at least about 60 degrees of the ring 200, or to a different amount as desired. In one example, the entirety of the ring 200 may overlap such that the overlapping portions 214, 216 comprise the entirety of the ring, for example, about 360 degrees, or even greater than 360 degrees. In one example, the ring 200 may be configured to have a single overlap, as shown in FIG. 2A, which may reduce the amount of material comprising the ring 200 and may ease the transition between the linearized configuration and the ring-shaped configuration. As will be apparent in the discussion below, the degree of overlap can change when the ring is in use. For example, tensioning and/or applying a load to the cover can reduce a diameter/circumference of the ring, thereby increasing the overlap in some examples.

The ring 200 may have a thickness 222 (in the axial dimension 218) and may have a width 224 (in the radial dimension 220) (as marked in FIG. 2A). The thickness 222 may be between about 0.2 and about 0.4 millimeters, although in other examples other thicknesses 222 may be utilized. In one example, the thickness 222 may be about 0.3 millimeters. In some examples, the thickness can be non-uniform along a length/circumference of the ring. For example, in some rings, at least one of the overlapping portions can be thinner than a non-overlapping portion of the ring. The width 224 may be between about 0.3 and about 0.5 millimeters, although in other examples other widths 224 may be utilized. In one example, the width 224 may be about 0.4 millimeters. In some examples, the width is non-uniform along a length/circumference of the ring, for example, wider at at least one of the overlapping portions. The ring 200 may be sized as desired, and may be configured to be a relatively thin ring that is flexible to allow for ease of movement of the ring 200.

Referring to FIG. 2B, the ring 200 may have a flattened shape with a substantially planar top surface 226 and a substantially planar bottom surface 228 facing opposite the top surface 226. The ring 200 at the overlapping portions 214, 216 may have the top surface 226 of portion 216 face towards the bottom surface 228 of portion 214. The terms “top” and “bottom” may be used interchangeably. Side surfaces 230 may connect the top surface 226 to the bottom surface 228. The body of the ring 200 may have a rectangular profile when viewed in cross section.

The ring 200 may be made of a material that is flexible, such that the ring may move from the linearized configuration (as shown in FIG. 2H) to the ring-shaped configuration (as shown in FIG. 2A). The ring 200 may be made of an elastic material to move from one configuration to the other relaxed or default configuration. In one example, the ring 200 may be made of a super elastic or shape-memory material, which may include a shape-memory alloy, to allow the ring to move from the linearized configuration to the ring-shaped configuration. The shape-memory material may be a material such as nitinol or another shape-memory material. The ring 200 may be configured to automatically move from the linearized configuration to the ring-shaped configuration, as the shape-memory material may automatically move to the shape-set ring-shaped configuration.

A linearized configuration is shown in FIG. 2H. In this configuration, the portions 214, 216 of the ring 200 may be separated from each other and do not overlap. The ends 204, 206 of the ring 200 do not overlap. The ring 200 in FIG. 2H may be in one form of linearized configuration, however, other forms may be utilized. For example, two opposite portions of the ring 200 in the ring-shaped configuration may be squeezed together towards a center portion of the ring such that the opposite portions of the ring-shaped body come together at the center portion and the ring 200 is linearized. Other forms of linearization may be utilized. The ring 200 in the linearized configuration may not be entirely straightened, although in other examples, the ring 200 may be entirely straightened (as shown in FIG. 2H). As such, the term “linearized” refers to any configuration that is suitable for delivery and deployment through a catheter or other minimally invasive or percutaneous delivery system, and does not require that the ring or any portion thereof is substantially straight or linear. In some examples, no portion of the ring is substantially straight or linear in the linearized configuration. For example, all or a portion of a ring can adopt a helical, sinusoidal, and/or other curved shape in the linearized configuration. Consequently, the terms “delivery configuration” and “open configuration” can also be used to describe some examples. The ring 200 in the linearized configuration may be configured to fit within the lumen of a deployment apparatus 303, as marked in FIG. 2H.

FIG. 2C illustrates a top view of the cover 212 unfolded. The cover 212 may include a top edge 232 and an opposite bottom edge 234. Side edges 236, 238 may extend from the top edge 232 to the bottom edge 234. The terms “top” and “bottom” may be used interchangeably.

The cover 212 may include a plurality of cut-outs 240 defining openings 242 in the cover 212. The cut-outs 240 may be in the form of a pattern of shapes. The shapes, as shown in FIG. 2C, may be asymmetric diamonds. The asymmetric diamonds may include opposite triangular portions 244, 246. The triangular portions 244 may have an angle and side lengths from the central vertex 248 that is different than the angle and side lengths from the central vertex 250 of the triangular portion 246. The angle from the central vertex 248 is greater than the angle from the central vertex 250, and the side lengths from the central vertex 248 are shorter than the side lengths from the central vertex 250. The side lengths of the triangular portions 244, 246 extend to straight side portions 252, 254.

The cut-outs 240 may leave the remaining portion of the cover 212 with trapezoidal portions 256, 258 having differing heights and side lengths. The height and side lengths of the trapezoidal portions 256 may be less than the height and side lengths of the trapezoidal portions 258. The trapezoidal portions 256, 258 may be connected with rectangular portions 260.

The pattern of cut-outs 240 may be repeated along the length of the cover 212. The shape and pattern of cut-outs 240 and shape and pattern of the remaining portions of the cover 212 may be varied from the shapes and pattern shown in FIG. 2C as desired. In some examples in which the ring has a non-circular ring-shaped or closed configuration, the particular details of the cover can differ, for example, the patterns of shapes defined by the cut-outs and/or their dimensions.

The cover 212 may include a fold portion 262 marked in dashed lines in FIG. 2C. The cover 212 may be configured to fold at the fold portion 262. The portion of the cover 212 shown above the fold portion 262 may comprise an overlapping portion 264 and the portion of the cover 212 shown below the fold portion 262 and above the dot-dashed line may comprise an overlapping portion 266. The overlapping portion 266 may overlap the overlapping portion 264 when the cover 212 is folded at the fold portion 262. The cover 212 may include a fold portion 263 marked in dot-dashed lines in FIG. 2C. The cover 212 may include an overlapping portion 268 indicated below the dot-dashed line in FIG. 2C. The overlapping portion 268 may be configured to overlap the top edge 232 of the cover 212 and a portion of overlapping portion 264 when the cover 212 is folded upon the fold portion 262.

The dimensions of the cover 212 may be set as desired. The dimensions may include a length 270. The length 270 may extend from the side edge 236 to the side edge 238. The length 270 may be between about 100 millimeters and about 70 millimeters, and in other examples, may have a greater or lesser size as desired. In one example, the length 270 may be about 88 millimeters. The dimensions may include a width 272 of the unfolded cover 212. The width 272 may extend from the top edge 232 to the bottom edge 234. The width 272 may be between about 20 millimeters and about 30 millimeters, and in other examples, may have a greater or lesser size as desired. In one example, the width 272 may be about 22 millimeters.

The dimensions may include a width 274 of the cover 212 from the bottom edge 234 to the lower end of the cut-outs 240. The width 274 may be between about 4 millimeters and about 7 millimeters, and in other examples, may have a greater or lesser size as desired. In one example, the width 274 may be about 5.5 millimeters. The dimensions may include a width 275 of the cut-outs 240. The width 275 may be between 10 about millimeters and about 15 millimeters, and in other examples, may have a greater or lesser size as desired. In one example, the width 275 may be about 13.5 millimeters. The dimensions may include a width 276 of the rectangular portions 260. The width 276 may be between about 3 millimeters and about 7 millimeters, and in other examples, may have a greater or lesser size as desired. In one example, the width 276 may be about 5 millimeters. The dimensions may include a width 277 of the cover 212 from the top edge 232 to the upper end of the cut-outs 240. The width 277 may be between about 1 millimeter and about 5 millimeters, and in other examples, may have a greater or lesser size as desired. In one example, the width 277 may be about 3 millimeters.

The dimensions may include a thickness 278 of the rectangular portions 260. The thickness 278 may be between about 1 millimeter and about 5 millimeters, and in other examples, may have a greater or lesser size as desired. In one example, the thickness 278 may be about 3 millimeters. The dimensions may include a thickness 279 of the cut-outs 240. The thickness 279 may be between about 3 millimeters and about 8 millimeters, and in other examples, may have a greater or lesser size as desired. In one example, the thickness 279 may be about 6 millimeters.

FIG. 2D illustrates the cover 212 having been folded at the fold portion 262. The overlapping portion 266 overlaps the overlapping portion 264 (and the overlapping portion 264 overlaps the overlapping portion 266). The overlapping portions 264, 266 form respective layers, including a first layer 280 and a second layer 282 that overlap and may be in contact with each other. The cover 212 may be folded such that the central vertices 250 may be brought towards the central vertices 248, and the trapezoidal portions 258 overlap the rectangular portions 260. The triangular portions 246 of the cut-outs may form triangular-shaped gaps between the trapezoidal portions 258. The cover 212 in this configuration includes a plurality of protrusions 247 extending from a connecting portion 249 of the cover 212.

The cover 212 at the fold portion 262 may form a coupler 284 for coupling the cover 212 to a tension member 286 (as shown in FIG. 2G). The cover 212 at the fold portion 263 may form a coupler 287 (marked in FIG. 2E) for coupling to the ring 200. The couplers 284, 287 may comprise folded material at the fold portions 262, 263 that the respective tension member 286 and ring 200 may be passed through.

FIG. 2E illustrates a side view of the cover 212 in the configuration shown in FIG. 2D. The position of the tension member 286 (if coupled to the cover 212) is shown in dashed lines at the top of the cover 212 and the position of the ring 200 (if coupled to the cover 212) is shown in dashed lines at the bottom of the cover. The overlapping layers 280, 282 are visible. The overlapping portion 268 overlaps the edge 232 of the cover 212. The overlap of the overlapping portion 268 forms the coupler 287 in the form of a loop at a bottom end of the cover 212. The bottom end, when the ring 200 is in the ring-shaped configuration, may comprise a peripheral portion of the cover 212. Connectors 288 may extend through the layers 280, 282 and overlapping portion 268 to secure the loop in position at the bottom end of the cover 212. The connectors 288 may comprise sutures or other form of stitching, or another form of connector that connects the overlapping layers 280, 282 and overlapping portion 268. The connectors 288 may pass through the openings 208, 210 in the ring 200 to securely connect the ring 200 to the cover 212. The ring 200 may be positioned between the connectors 288 and the fold portion 263, and sandwiched between the layers 280, 282.

The cover 212 at the fold portion 262 forms a coupler 284 in the form of a loop at the top end of the cover 212. The top end, when the ring 200 is in the ring-shaped configuration, may comprise a central portion of the cover 212. The tension member 286 may pass through the coupler 284 and may be sandwiched between the layers 280, 282.

FIG. 2F illustrates an example of a cover 281 having a different configuration of cut-outs than shown in the example of FIGS. 2C-2E, 2G and 2H. The cut-outs in the example of FIG. 2F are symmetrical as folded upon the fold portion 289. The remaining portions of the cover 281 include a first layer 290 and a second layer 291 that have the same symmetrical shapes of trapezoidal portions coupled to rectangular portions (that include the fold portion 289). An overlapping portion 292 may overlap the layers 290, 291 and may form a coupler for coupling to the ring 200, in a similar manner as the example of FIGS. 2C-2E, 2G and 2H. The fold portion 289 may form a coupler for coupling to the tension member 286, in a similar manner as the example of FIGS. 2C-2E, 2G and 2H. The configuration of cover 281 may be utilized with the systems and methods disclosed herein, in a similar manner as cover 212. The shape and configuration of the covers 212, 281 shown in FIGS. 2C-2H may be varied as desired.

The covers 212, 281 may be flexible and configured to move with the ring 200 as it moves from the linearized configuration to the ring-shaped configuration. The covers 212, 281 may be made of a flexible material, which may include, for example, a cloth or fabric. The flexible material may be woven or non-woven. The flexible material may include materials such as ultra-high-molecular-weight polyethylene (UHMwPE) (for example, DYNEEMA® fabric or laminate, Koninklijke DSM, the Netherlands) or polyethylene terephthalate (PET, for example, DACRON® fabric, Invista, Wilmington, Delaware). In other examples, other flexible materials may be utilized.

FIG. 2G illustrates the ring 200 in a ring-shaped configuration, with the ring 200 coupled to the cover 212, and the tension member 286 coupled to the cover 212. Portions of the ring 200 coupled to the cover 212 may overlap, in a manner discussed previously. Portion 245 comprises an overlapping portion of the ring 200 and the cover 212. The cover 212 extends inward from the ring 200 in the ring-shaped configuration.

The tension member 286 is coupled to the cover 212 at the fold portions 262. The tension member 286 is drawn away from the cover 212 such that the cover 212 is drawn towards a central opening 293 of the cover 212. The tension member 286 accordingly may cinch the cover 212 towards the central opening 293. In some examples, the cover 212 can in turn pull on the ring 200, reducing a diameter/circumference thereof. The cover 212 is in a disc-shaped configuration. The cover 212 in this configuration includes a central portion 294 and a peripheral portion 295. The overlapping layers of material of the cover 212 (the layers 280, 282) extend from the peripheral portion 295 to the central portion 294. The fold portions 262 are positioned at the central portion 294, and the ring 200 is positioned in the peripheral portion 295.

The trapezoidal portions 258 of the layer 280 may be placed adjacent each other such that the gaps between the trapezoidal portions 258 shown in FIG. 2D are closed. The cover 212 accordingly may comprise a closed surface extending from the peripheral portion 295 to the central portion 294. The protrusions 247 are adjacent each other and extend from the peripheral portion 295 to the central portion 294.

The tension member 286 may comprise a portion of a heart splint, and may be configured to provide tension between the anchors of a heart splint. The tension member 286 may comprise a tether, and may be in the form of a cord, or other form of tension member. The tension member 286 may be made of a flexible material, which may include ultra-high-molecular-weight polyethylene (UHMwPE) (for example, FORCE FIBER® suture, Teleflex, Wayne, Pennsylvania or DYNEEMA® fiber, Koninklijke DSM, the Netherlands), among other flexible materials. The tension member 286 may include a body, and may include a coupling device 296 at its end that may couple the tension member 286 to itself. The coupling device 296 may comprise a loop that the body of the tension member 286 passes through, such that as the body of the tension member 286 is pulled, a size of a loop 297 formed by the tension member 286 being threaded through the fold portions 289 of the cover 212 reduces in size. The portion of the tension member 286 forming the loop 297 passes through the coupler 284 (marked in FIG. 2D) positioned at the central portion 294 of the cover 212. Thus, as the tension member 286 is pulled, the size of the loop 297 reduces, and accordingly the cover 212 is cinched and pulled radially towards the central opening 293 of the cover 212. The anchor 202 in the configuration shown in FIG. 2G may have a diameter of between about 20 millimeters and about 25 millimeters, although other diameters may be utilized as desired. In one example, the anchor 202 may have a diameter of about 22 millimeters.

The cover 212 may be configured to be drawn towards the central opening 293 such that the central opening 293 entirely closes. The tension member 286 may extend from the cover 212 at the central portion 294 of the cover 212.

FIG. 2H illustrates the ring 200 in a linearized configuration. The ring 200 is extended such that the ends 204, 206 are separated from each other. The tension member 286 is visible extending through the coupler 284 of the central portion 294. The anchor 202 is in a linearized configuration.

A deployment member 301 may be utilized to deploy the cover 212 of the anchor 202. The deployment member 301 may comprise a tether, and may be in the form of a cord, or other form of deployment member. The deployment member 301 may be looped, and may be coupled to the cover 212 at the coupler 284. The deployment member 301 may be looped through the coupler 284 in a manner shown in FIG. 2H. The deployment member 301 may be pulled to cinch or draw the cover 212 towards the central opening 293 of the cover 212 in a similar manner as discussed above regarding the tension member 286. The anchor 202, upon being positioned in the lumen of a deployment apparatus, may have the cover 212 flattened. The deployment member 301 may close or otherwise cinch the cover 212. The anchor 202 may be held against the end of the deployment apparatus 303 when the deployment member 301 is pulled, to support the anchor 202 in position. The ring 200, however, may also be configured to automatically move to or towards the ring-shaped configuration.

The anchor 202 may accordingly be configured to move from an unexpanded configuration to an expanded configuration. The unexpanded configuration may comprise the configuration in which the ring 200 is in the linearized configuration, and the anchor 202 is accordingly linearized. The expanded configuration may be the configuration in which the ring 200 is in the ring-shaped configuration and the cover 212 is in a disc-shaped configuration. In other examples, other unexpanded and expanded configurations may be utilized. In an expanded configuration, an anchor may have a larger diameter or other dimensions. In an unexpanded configuration, the anchor may have a smaller diameter or other dimensions and may be configured to fit within the lumen of a deployment apparatus. The configurations of anchor may vary from that shown in FIGS. 2G and 2H.

The anchor 202 may beneficially be configured such that the cover 212 bears the majority of the force against the anchor 202 when the tension member 286 is tensioned. The ring 200 may be configured to provide support for the shape of the cover 212, but otherwise may bear a lesser portion of the force against the anchor 202. The overlapping portions of the ring 200 may beneficially provide enhanced strength for the ring 200.

The relatively thin shape of the ring 200 may allow the ring 200 to be flexible to fit within the lumen of a deployment apparatus. The ring 200 may be able to be positioned within the lumen of a deployment apparatus with a relatively low force, and may be positioned within the lumen manually. The ring 200 may be sufficiently flexible to be hand-loaded into a deployment apparatus.

The anchor 202 may have a variety of uses, including use as a portion of a heart splint as may be disclosed herein.

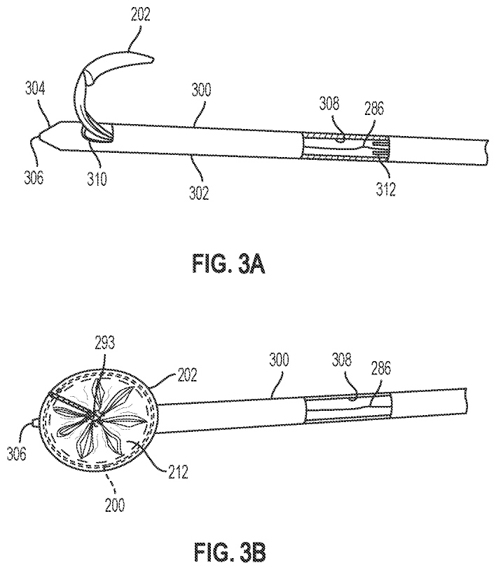

FIG. 3A illustrates an example of a deployment apparatus 300 that may be utilized according to examples herein. The deployment apparatus 300 may comprise a puncturing device include a body portion 302 and a distal end 304. The deployment apparatus 300 may be utilized in a method of applying heart anchors to a patient's heart.

The distal end 304 of the deployment apparatus 300 may include a puncturing tip 306. The puncturing tip according to examples herein may be for puncturing one or more surfaces of a heart, including an external posterior surface and an external anterior surface of the heart. The deployment apparatus 300 may include an interior lumen 308 and may include an opening 310 along the body portion 302 positioned adjacent the puncturing tip 306. The interior lumen 308 may comprise an implant retention area for retaining a heart anchor in an unexpanded configuration. The deployment apparatus 300 may include a push device 312 for passing through the lumen 308 for pushing a heart anchor 202 out of the opening 310.

The body portion 302 may have the shape of an elongate rigid rod. The body portion 302 may be sufficiently rigid to withstand the force of penetrating through a portion of a patient's heart.

The push device 312 may be configured to pass through the lumen 308 with an internal lumen to allow the tension member 286 to pass through the lumen 308 and the internal lumen, and be accessible for tensioning by a user.

The interior lumen 308 may be configured to retain the anchor 202 in the unexpanded or linearized configuration within the lumen 308. The anchor 202 may be positioned within the lumen 308 such that as the anchor 202 is pushed out of the lumen 308 with the push device 312, the tension member 286 remains in the lumen 308 and a portion of the tension member 286 remains accessible to be pulled to move the cover 212 towards the central opening 293 of the cover 212 as discussed herein. The ring 200 (marked in FIG. 2G) of the anchor 202 may be configured to automatically move to the ring-shaped configuration, as discussed herein. In some examples, a separate deployment member 301 as shown in FIG. 2H may be utilized to be pulled to move the cover 212 towards the central opening 293 of the cover. However, in some examples, the tension member 286 itself may be pulled.

The anchor 202 may be configured to move from the unexpanded configuration to the expanded configuration adjacent the opening 310. In one example, multiple anchors 202 may be positioned within the lumen 308 and may be pushed out in sequence.

FIG. 3B illustrates the anchor 202 passed out of the opening 310 and in the expanded configuration in which the ring 200 (marked in FIG. 2G) of the anchor 202 is in the ring-shaped configuration.

FIGS. 4A-4E illustrate systems, apparatuses, and methods according to examples herein. FIGS. 4A-4E may represent steps in a method of approximating papillary muscles of a heart.

Referring to FIG. 4A, the heart 100 may be suffering from a dilated left ventricle 102, which may comprise ischemic or non-ischemic dilated cardiomyopathy, as represented for example in FIG. 1B. Chordal tethering may result, and the mitral valve 118 may be suffering from functional mitral regurgitation. The methods as disclosed herein may be for approximating papillary muscles of the heart to treat the functional mitral regurgitation, among other maladies.

As shown in FIG. 4A, the heart 100 may be penetrated from anterior to posterior. The anterior wall 104 may be penetrated with a deployment apparatus 300, which may be configured similarly as shown in FIGS. 3A and 3B. The external anterior surface 401 may first be punctured with the puncturing tip 306 and the body portion 302 may extend through the interior cavity 108 to the posterior wall 106. The puncture site on the external anterior surface 401 may be proximate the papillary muscles 120, and particularly proximate the anterior lateral papillary muscles. The deployment apparatus 300 may pass through the papillary muscles 120 proximate the anterior wall 104 or adjacent to such papillary muscles 120 in some examples.

Access to the anterior wall 104 of the heart 100 may be provided with a sternotomy, a thoracotomy, or a mini thoracotomy, among other access methods.

The anterior wall 104 may be penetrated on a beating heart, either with or without rapid pacing, or on an arrested heart in some examples. The entire procedure of deploying the heart splint, and approximating the papillary muscles, or a portion of the procedure, may occur on a beating heart, either with or without rapid pacing, or on an arrested heart in some examples. Rapid pacing of the heart may be performed at certain portions of the procedure to minimize heart motion at certain steps. In some examples, the entire procedure may occur while rapid pacing the heart. Performing a puncturing step on a rapidly-paced heart 100 herein may confer an advantage of minimizing heart motion and make targeting the appropriate location with medical imaging (such as echocardiography (including transesophageal echocardiography (TEE)) easier.

Echocardiography (including transesophageal echocardiography (TEE)) or other forms of medical imaging may be utilized to determine the desired puncture site on the anterior wall. Such medical imaging may further be utilized to determine a puncture site on the posterior wall 106 of the heart 100, proximate the papillary muscle 121 at the posterior wall 106. Such papillary muscles may comprise posterior medial papillary muscles. A desired trajectory from the puncture site on the anterior wall 104 to the puncture site on the posterior wall 106 may be determined utilizing medical imaging.

In some examples, a shield 402 may be advanced around the posterior side of the heart 100. The shield 402 may be deployed to prevent the deployment apparatus 300 from piercing a lung after exiting the posterior wall 106 of the heart 100. The shield 402 in some examples may comprise an expandable shield, made for example of a mesh made of shape memory material. The shape memory material in some examples, may comprise nitinol or other forms of shape memory material.

The deployment apparatus 300 may be passed through the left ventricle 102 to the posterior wall 106. Such movement may be visualized using medical imaging as discussed herein. The puncture site on the posterior wall 106 may be on the interior surface of the posterior wall 106, proximate the papillary muscles 121 proximate the posterior wall 106. The puncturing tip 306 may puncture through the posterior wall 106 and extend out the external posterior surface of the heart 100. The deployment apparatus 300 may be passed through the papillary muscles 121 proximate the posterior wall 106 or adjacent to such papillary muscles 121 and through the external posterior surface 403. The opening 310 of the deployment apparatus 300 may be positioned exterior of the external posterior surface 403.

The deployment apparatus 300 punctures the external anterior surface 401 of the heart 100 and extends across the interior cavity 108 of the left ventricle 102 from the external anterior surface 401 to the external posterior surface 403 of the heart 100.

With the deployment apparatus 300 extending in an anterior-posterior direction of the heart 100, and through the left ventricle 102, a heart anchor 202 as shown in FIGS. 3A and 3B, for example, may be deployed from the implant retention area and the opening 310. The deployment apparatus 300 may be configured to deploy the heart anchor 202 from an unexpanded configuration to an expanded configuration at the external posterior surface 403 of the heart 100. The heart anchor 202 may be moved from the unexpanded configuration to an expanded configuration at the external posterior surface 403 of the heart loft A push device 312, for example as shown in FIG. 3A, may push the unexpanded heart anchor 202 out of the opening 310 at a position exterior of the external posterior surface 403 of the heart 100. FIG. 4A, for example, illustrates the heart anchor 202 being deployed from the deployment apparatus 300. In some examples, spacers such as retractors, balloons, or other devices may be utilized to create sufficient space on the posterior side of the heart 100 for the heart anchor 202 to be deployed.

FIG. 4B illustrates the heart anchor 202 fully deployed at the external posterior surface 403 of the heart 100. The deployment apparatus 300 may continue to extend from the anterior wall 104 to the posterior wall 106 across the interior cavity 108. With the heart anchor 202 fully deployed, the deployment apparatus 300 may be retracted, leaving the deployed heart anchor 202 to abut the puncture site at the external posterior surface 403 of the heart 100. The heart anchor 202 may be deployed to the external posterior surface 403 of the heart 100 proximate the papillary muscle 121 of the heart 100.

The tension member 286 may trail from the opening 310 of the deployment apparatus 300 (as shown in FIG. 4C for example) as the deployment apparatus 300 is retracted proximally in the anterior direction from the puncture site on the posterior wall 106. The heart anchor 202 may be coupled to a distal end of the tension member 286. The tension member 286 accordingly may extend from the coupling to the heart anchor 202, through the posterior wall 106, across the interior cavity 108, and out the puncture site on the anterior wall 104.

FIG. 4C illustrates the deployment apparatus 300 having been retracted proximally and out the anterior wall 104 of the heart 100. The tension member 286 is trailing, and extends across the interior cavity 108 from the posterior wall 106 to the anterior wall 104. A proximal portion of the tension member 286 extends out of the puncture site on the external anterior surface 401. The proximal portion of the tension member 286 is accessible at the anterior side of the heart 100.

FIG. 4D illustrates that a second, or another, heart anchor 404 may be engaged with the proximal portion of the tension member 286 and advanced distally to abut the external anterior surface 401 of the heart 100. The heart anchor 404 may comprise a pad that is configured to distribute force upon the external anterior surface of the heart 100 upon a wider surface area than provided by the puncture site on the external anterior surface 401.

The heart anchor 404 may include a lock 538 for locking the tension member 286 to the heart anchor 404. The lock 538 may comprise a releasable lock, that allows the lock to release if desired.

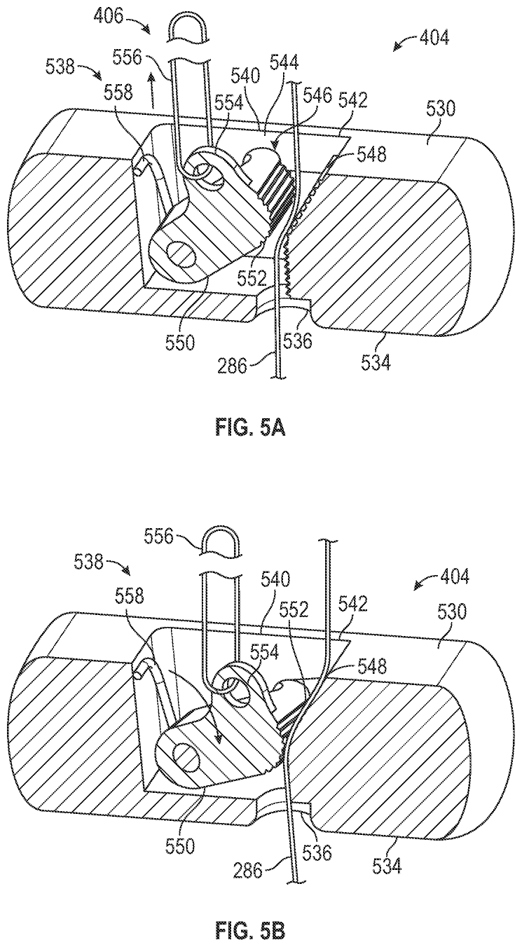

FIG. 5A, for example illustrates a cross sectional view of an example of the heart anchor 404. A cross sectional view showing the lock 538 and a receiver 540 is provided. The receiver 540 may be configured to receive the proximal portion of the tension member 286.

The receiver 540 may include an opening 542 in the top surface 530 of the heart anchor 404 and may include the opening 536 in the bottom surface 534 of the heart anchor 404. The receiver 540 may include one or more side walls 544 that define a cavity 546 in the heart anchor 404. One of the side walls 544 may include a locking surface 548. The locking surface 548 may comprise a surface for the tension member 286 to be pressed against upon operation of the lock 538. The locking surface 548 may include a grip surface, which may include ridges or another gripping structure that may improve a grip of the locking surface 548.

The lock 538 may be positioned within the receiver 540. The lock 538 may be coupled to the heart anchor 404. The lock 538 may be configured to vary from an unlocked state in which the tension member 286 is unlocked in the receiver 540 to a locked state in which the tension member 286 is locked in the receiver 540. The lock 538 may be configured to move from the locked state to the unlocked state.

The lock 538 may include a rotatable body 550, which may be configured to rotate about a pivot. The pivot may comprise an axle extending through the rotatable body 550, or another form of pivot. The rotatable body 550 may be configured to rotate within the cavity 546 of the receiver 540. The rotatable body 550 may comprise a cam body with a surface of the body 550 comprising a locking surface 552. The cam body may allow the force from the lock 538 against the tension member 286 to increase as tension is increased upon the tension member 286. The lock 538 may comprise a cam-lock in some examples. The locking surface 552 may comprise a surface to press against the tension member 286, and press the tension member 286 against the locking surface 548, to lock the tension member 286 in position within the receiver 540. The locking surface 552 may include a grip surface, which may include ridges or another gripping structure that may improve a grip of the locking surface 552.

The lock 538 as such may include a ratchet mechanism, that may be configured to allow the tension member 286 to be drawn through the heart anchor 404 in a direction, and resist movement of the tension member 286 in an opposite direction. As such, the tension member 286 may be drawn in a proximal direction to tension the tension member 286, with the lock 538 resisting the tension member 286 from loosening in the distal direction.

In some examples, the lock 538 may include a connector 554 as shown in FIGS. and 5B, for example, for coupling with a lock retainer member 556. The connector 554 may include an aperture for the lock retainer member 556 to be passed through. The lock retainer member 556, for example, may comprise a tether such as a looped cord that couples to the connector 554. The lock retainer member 556 may be tensioned by a user to release the lock 538, and may be released or cut to set the lock 538.

The lock 538 may include a biasing device 558. The biasing device 558 may bias the lock 538 to a locked state, in which the rotatable body 550 is pressed towards the locking surface 548. The rotatable body 550 in the locked state may press the tension member 286 against the locking surface 548 to prevent movement of the tension member 286 and to lock the tension member 286 to the anchor 404. The biasing device 558 may comprise a spring, or other form of biasing device as desired.

The lock retainer member 556 may be pulled to oppose the biasing force of the biasing device 558 and may retain the lock 538 in an unlocked state. The lock retainer member 556 may be configured to couple to the rotatable body 550 to hold the rotatable body 550 in the unlocked state. Such an unlocked state is shown in FIG. 5A. The locking surface 552 of the rotatable body 550 is pulled away from the locking surface 548 of the receiver 540 and the tension member 286 may slide within the receiver 540 and through the openings 536, 542.

The tension member 286 may be slid within the receiver 540 to tension the tension member 286 before the lock 538 is moved to the locked state. Upon a desired amount of tension being reached, the lock retainer member 556 may be moved. The movement of the lock retainer member 556 towards the anchor 404 may allow the biasing device 558 to move the lock 538 to the locked state.

The heart anchor 404 may comprise a pad in which the bottom surface 534 forms a wide contact surface for contact with the external surface of the heart 100, such as the external anterior surface 401. The heart anchor 404 may have a disk shape, or may have other shapes as desired. The heart anchor 404 may be configured to have a static size that does not move from an unexpanded configuration to an expanded configuration. In some examples, the heart anchor 404 may be configured to move from an unexpanded configuration to an expanded configuration. Variations in the lock 538 and the heart anchor 404 may be provided in some examples.

Referring back to FIG. 4D, the heart anchor 404 may be deployed to the external anterior surface 401 proximate the papillary muscle 120 of the heart 100 with the proximal portion of tension member 286 passing through the lock 538. The proximal portion of the tension member 286 may be coupled to the heart anchor 404. The proximal portion of the tension member 286 may be drawn through the lock 538 with the heart anchor 404 advancing distally towards the external anterior surface 401. The heart anchor 404 may abut the external anterior surface 401, with the bottom surface 534 contacting the external anterior surface 401. The retraction of the tension member 286 may further cause the heart anchor 202 to be drawn to and contact the external posterior surface 403.

In the configuration shown in FIG. 4D, the tension member 286 for coupling the heart anchors 202, 404 may be tensioned to approximate the papillary muscles. Such tensioning of the tension member 286 may be applied during a beating heart procedure, or other procedure such as on an arrested heart. The tension applied to the tension member 286 in some examples may be titrated to achieve a desired amount of papillary muscle approximation, which may be assessed on a beating heart using medical imaging, including echocardiography. Such titration methods may be disclosed in U.S. patent application Ser. No. 16/549,957, titled METHODS AND DEVICES FOR VENTRICULAR RESHAPING AND HEART VALVE RESHAPING, filed on Aug. 23, 2019 and published as U.S. Publication No. 2020/0069426, the entire contents of which are incorporated herein. Deployment apparatuses, for example, may be utilized to titrate the tension.

The arrows shown in FIG. 4D represent the papillary muscle approximation, as the papillary muscles are displaced towards each other. The heart anchors 404, 202 may apply a compressive force to the respective external anterior surface 401 and external posterior surface 403 to approximate the papillary muscles 120, 121.

In a beating heart procedure, a user, such as a surgeon may be able to determine the variation in size of the ventricle, and may also determine the flow through the mitral valve 118 as the tension member 286 is being tensioned, via medical imaging or another form of visualization, or another method. The user may tension the tension member 286 until a desired amount of approximation occurs. If the tension member 286 is over-tensioned, then in some examples, the lock 538 may be released to allow the tension to be relieved and reset by the user. The tension member 286 may be locked in tension between the heart anchors 404, 202 across the ventricle 102.

The heart anchors 404, 202 and tension member 286 may form a heart splint 400 for approximating the papillary muscles 120, 121. The heart anchor 202 is configured to be positioned on the external posterior surface 403 of the heart 100 proximate the papillary muscle 121 of the heart 100. The heart anchor 404 is configured to be positioned on an external anterior surface 401 of the heart 100 proximate the papillary muscle 120 of the heart 100. The tension member 286 is configured to couple the heart anchors 404, 202 to each other and extend within the ventricle of the heart 100. The heart anchors 404, 202 may each comprise pads configured to distribute a compressive force along a surface area of the respective surfaces 401, 403.

The approximation of the papillary muscles 120 may reduce the possibility of chordal tethering, and improve the coaptation of the leaflets of the mitral valve 118.

FIG. 4E illustrates a top cross sectional view of the left ventricle 102 with the heart anchors 202, 404 in position and the tension member 286 in tension. Each heart anchor 404, 202 is deployed proximate the respective papillary muscles 120, 121 of the anterior wall 104 and the posterior wall 106. In some examples, the puncture site on the anterior wall 104 and posterior wall 106 may be through the papillary muscles 120, 121 as shown in FIG. 4E, although other locations may be utilized as desired such as adjacent to the papillary muscles 120, 121. Approximating the papillary muscles 120 may reduce the tension upon the chordae 122 and improve operation of the native valve.

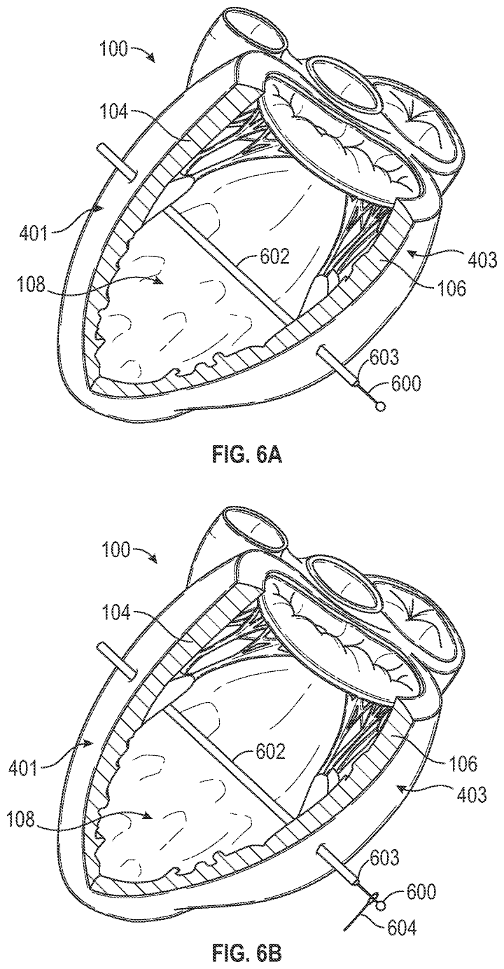

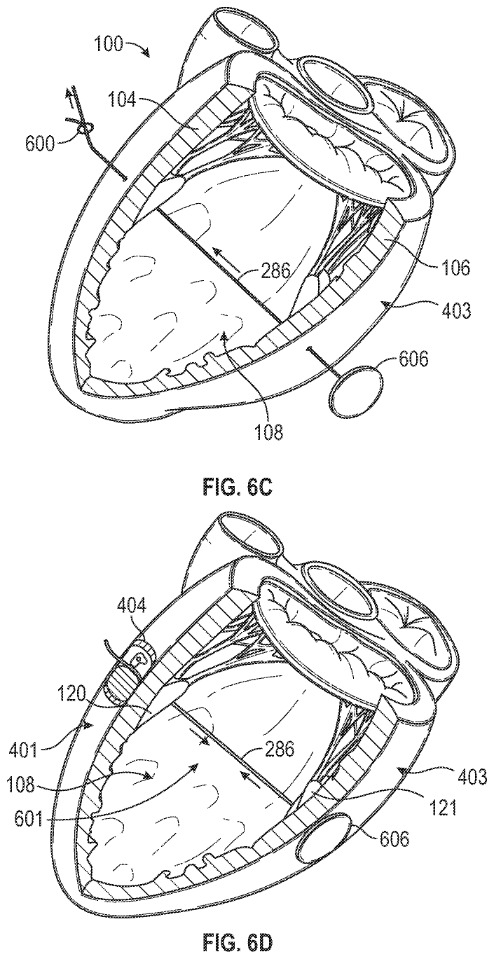

Variations in the methods disclosed herein may be provided. FIGS. 6A-6D, for example, illustrate an example in which a puncturing device in the form of a needle 602 or another form of relatively narrow catheter that may have a puncturing tip 603 is utilized. The needle 602 may include an interior lumen that may be configured to pass a snare 600 therethrough. The snare 600 may be configured to be deployed from the interior lumen of the needle 602. The snare 600 for example, may comprise a loop snare, having a loop at a distal end of the snare 600, or another configuration of snare may be utilized as desired.

In an example utilizing a snare 600 and a relatively narrow catheter such as a needle 602, a heart anchor may not need to be positioned in an unexpanded state in a deployment apparatus. As such, the puncture size of the anterior wall 104 and the posterior wall 106 may be reduced as the size (e.g., diameter) of the puncturing device may be reduced.

The needle 602 may be configured to puncture the external anterior surface 401, pass across the interior cavity 108, and puncture the external posterior surface 403 in a similar manner as the deployment apparatus 300 described in regard to FIGS. 4A-4B. The needle 602 may extend across the interior cavity 108 of the ventricle from the external anterior surface 401 to the external posterior surface 403. The size of the puncture provided by the needle 602 however, may be smaller than the size of the puncture provided by the deployment apparatus 300, as the needle 602 may not need to retain an unexpanded heart anchor therein and thus may have a lesser diameter than the deployment apparatus 300.

As the needle 602 extends out of the external posterior surface 403 as shown in FIG. 6A, the snare 600 may be configured to pass through the interior lumen of the needle 602 and out of the posterior wall 106 of the heart 100. The snare 600 may extend from the interior cavity 108 to be deployed external of the external posterior surface 403.

Referring to FIG. 6B, the snare 600, in some examples, may be configured to engage another snare 604 at the posterior side of the heart 100. With the snare 600 captured, the snare 600 may be engaged with a tension member 286 that is coupled to a heart anchor 606. The snare 600 may be withdrawn proximally across the interior cavity 108, drawing the tension member 286 proximally as well. The snare 600 may extend the tension member 286 across the interior cavity 108 of the ventricle.

FIG. 6C, for example, illustrates the snare 600 being drawn proximally, with the tension member 286 extending across the interior cavity 108. The movement of the tension member 286 may draw the heart anchor 606 to the external posterior surface 403 of the heart 100.

The heart anchor 606, in some examples, may be configured as a pad that contacts the external posterior surface of the heart 100. The pad may have a disk shape or another shape for distributing force upon the external posterior surface of the heart 100. The heart anchor 606 in some examples may be covered with a material such as cloth, that may form an outer surface of the heart anchor 606. The heart anchor 606 may otherwise be a rigid or flexible body. The heart anchor 606 may include a coupling point for coupling with the tension member 286.

The heart anchor 606, in some examples, may have a static size, and may not be configured to expand from an unexpanded configuration to an expanded configuration. In some examples, the heart anchor 606 may be configured to expand from an unexpanded configuration to an expanded configuration.

Referring to FIG. 6D, the proximal portion of the tension member 286 may engage another heart anchor 404. The heart anchor 404 may operate similarly as discussed in regard to FIGS. 5A-5B and 4D. The tension in the tension member 286 may be set to a desired amount, and the heart anchor 404 may be locked to set the tension in the tension member 286 in a similar manner as discussed regarding the examples of FIGS. 4A-5B.

The heart anchors 404, 606 and tension member 286 may form a heart splint 601 for approximating the papillary muscles 120, 121. The heart anchor 606 is configured to be positioned on the external posterior surface 403 of the heart wo proximate the papillary muscle 121 of the heart 100. The heart anchor 404 is configured to be positioned on an external anterior surface 401 of the heart 100 proximate the papillary muscle 120 of the heart 100. The tension member 286 is configured to couple the heart anchors 404, 202 to each other and extend within the ventricle of the heart 100. The heart anchors 404, 202 may each comprise pads configured to distribute a compressive force along a surface area of the respective surfaces 401, 403.

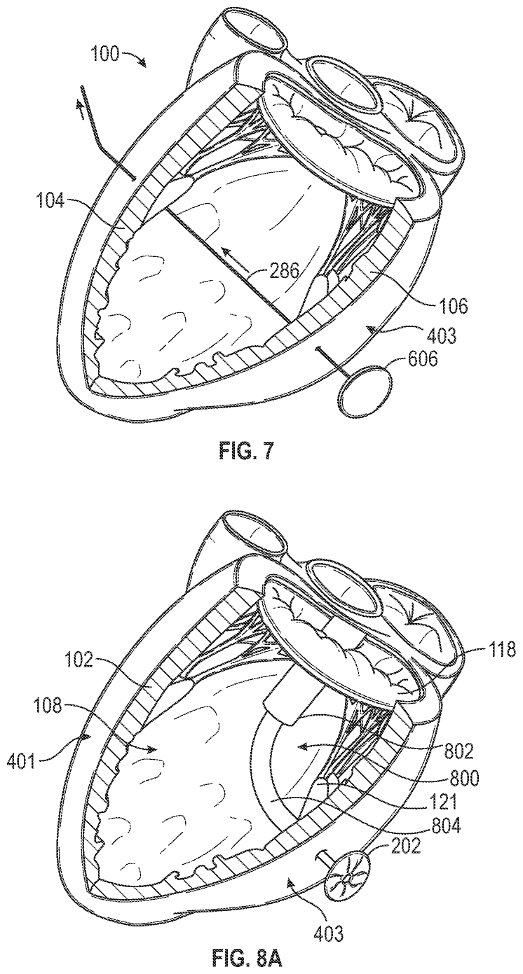

FIG. 7 illustrates a variation in the procedure of FIGS. 6A-6D, in which the snare 600 may be directly coupled to the tension member 286 and may pass the tension member 286 distally through the interior lumen of the needle 602. The snare 600 may then be snared to couple the distal end of the tension member 286 to the heart anchor 606. The distal end of the tension member 286 may be coupled to a coupling point on the heart anchor 606. The tension member 286 may then be withdrawn proximally, drawing the heart anchor 606 to the external posterior surface 403 of the heart 100. The snare 600 may be utilized to extend the tension member 286 across the interior cavity 108 of the ventricle. The second heart anchor 404 may then be utilized to couple to the proximal portion of the tension member 286 as described in regard to FIG. 6D. The resulting configuration may appear similar as the heart splint shown in FIG. 6D.

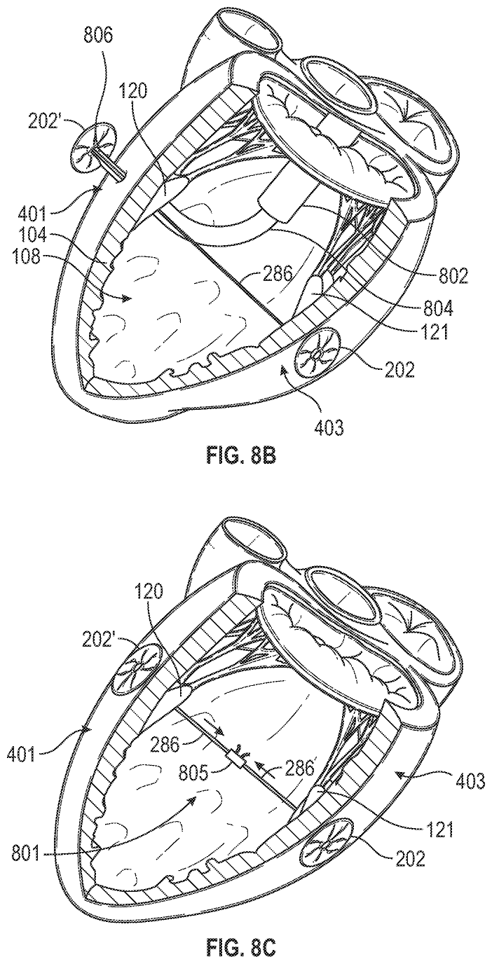

FIGS. 8A-8D illustrate an example in which transcatheter and/or transvenous methods may be utilized to deploy the heart splint. FIG. 8A, for example, illustrates a deployment apparatus 800 in the form of an elongate shaft including a sheath 802 and a steerable distal end 804. The steerable distal end 804 may extend from the sheath 802. In other examples, other configurations of elongate shafts may be utilized to pass through the vasculature of the patient's body.

The elongate shaft may be configured to pass from an atrium into the ventricle. The steerable distal end 804 may include a puncturing tip 806 (marked in FIG. 8B). The steerable distal end 804 may be configured to be steered to position the puncturing tip 806 to pass through the external posterior surface 403 to deploy a heart anchor at the external posterior surface 403 and may be configured to position the puncturing tip 806 to pass through the external anterior surface 401 to deploy a heart anchor at the external anterior surface 401.

The elongate shaft of the deployment apparatus 800 may be configured to pass through the mitral valve 118 and into the interior cavity 108 of the left ventricle 102. In some examples, the elongate shaft may be passed transseptally into the left atrium (e.g., via a transseptal puncture between the right atrium and left atrium) and then in a ventricular direction towards the interior cavity 108. In other examples, other access methods to the interior cavity 108 may be utilized.

The deployment apparatus 800 may be configured to puncture the posterior wall 106 and the anterior wall 104 either together or sequentially. FIG. 8A, for example, illustrates the steerable distal end 804 of the deployment apparatus 800 being steered towards the interior surface of the posterior wall 106. A puncturing tip 806 of the deployment apparatus 800 punctures the interior surface and deploys an expandable heart anchor, such as the heart anchor 202, to a position on the external posterior surface 403 of the heart proximate a papillary muscle 121 of the heart 100.

FIG. 8B, for example, illustrates the steerable distal end 804 being then directed to penetrate the interior surface of the anterior wall 104. The distal end 804 may be configured to be steerable in a variety of methods, including operation of one or more pull wires for steering the distal end 804, or via rotation of the distal end 804, or via another method as desired.

A heart anchor 202′ may be deployed on the external anterior surface 401 of the heart proximate the papillary muscle 120. Both heart anchors 202′, 202 may be configured similarly as each other in some examples. In some examples, either of the heart anchors 202′, 202 may have a different configuration than each other. The tension member 286 may trail between the heart anchors 202, 202′ and extend across the interior cavity 108.

The steerable distal end 804 may then be withdrawn into the interior cavity 108. The tension in the tension member 286 may be increased centrally, within the interior cavity 108. A lock 805 may be deployed between the portions of the tension member 286 and set, as shown in FIG. 8C for example. The tension in the tension member 286 may be set and controlled in a similar manner as discussed regarding FIG. 4D. Other forms of entry and locking methods that may be utilized may be disclosed in U.S. patent application Ser. No. 16/549,957, titled METHODS AND DEVICES FOR VENTRICULAR RESHAPING AND HEART VALVE RESHAPING, filed on Aug. 23, 2019 and published as U.S. Publication No. 2020/0069426, the entire contents of which are incorporated herein.

The heart anchors 202, 202′ and tension member 286 may form a heart splint 801 for approximating the papillary muscles 120, 121. The heart anchor 202 is configured to be positioned on the external posterior surface 403 of the heart 100 proximate the papillary muscle 121 of the heart 100. The heart anchor 202′ is configured to be positioned on an external anterior surface 401 of the heart 100 proximate the papillary muscle 120 of the heart 100. The tension member 286 is configured to couple the heart anchors 202′, 202 to each other and extend within the ventricle of the heart 100. The heart anchors 202′, 202 may each comprise pads configured to distribute a compressive force along a surface area of the respective surfaces 401, 403.

In some examples, other forms of transcatheter and/or transvenous approaches may be utilized to deploy the heart anchors. For example, a deployment apparatus may simultaneously deploy both heart anchors in some examples. In some examples, other forms of locking procedures may be utilized. Other approaches may be utilized in some examples, including transapical approaches and other transseptal approaches (e.g., across the interventricular septum). An approach may include passing the deployment apparatus through the aortic valve. In some examples, snares may be passed out of one or more of the external posterior surface 403 or the external anterior surface 401 to engage heart anchors for applying a compressive force to the respective surfaces.

In some examples, a beating heart procedure may allow a user, such as a surgeon, to better determine the effects of the approximation of the papillary muscles, to better determine the possible results of such a procedure. Such a method may comprise an improvement over methods of operating on an arrested heart, in which hemodynamics may not be monitored in real time. The systems, apparatuses, and methods disclosed herein, however, may be utilized on an arrested heart in some examples.

In some examples, the systems, apparatuses, and methods disclosed herein are disclosed in regard to approximation of the papillary muscles of the left ventricle. However, in some examples, approximation of papillary muscles of the right ventricle may be performed in a similar manner. Such approximation may address tricuspid valve regurgitation of the tricuspid valve. The heart anchors may be positioned on an external anterior surface and an external posterior surface of the right ventricle in such a manner, in a similar manner as disclosed herein.

The methods disclosed herein may beneficially provide for treating a dilated ventricle of the heart, while providing a minimally invasive procedure. Under the methods disclosed, a full sternotomy may not be required, and entry into the left ventricle may comprise an endovascular entry into the patient's heart. The application of the splint may comprise a beating-heart repair of the left ventricle. The method may include reshaping a ventricle of the heart by applying pressure to the heart to reshape the geometry of heart. Endovascular or transcatheter methods may be utilized. Percutaneous entry of the patient's body may occur. In one example, a full sternotomy may be performed if desired. The anchor 202 may be configured to easily deploy to the expanded configuration, and may be moved to the unexpanded or linearized configuration with a low force. The cover 212 of the anchor 202 may bear the majority of the force against the anchor 202 while the overlapping portions of the ring 200 may provide support to the ring 200. In some examples, multiple heart anchors, including more than two heart anchors (e.g., at least three, at least four, etc.), may be utilized.

The apparatuses and other components disclosed herein may comprise one or more systems. The systems may be utilized in a variety of methods. The methods may include the methods disclosed herein. The methods may include a method for treating ventricular dilation and/or mitral regurgitation and/or tricuspid regurgitation. The methods may include deploying a heart splint.

The steps disclosed herein are illustrative, and may be modified, varied, reordered, or excluded as desired. The “steps” referred to herein may include multiple steps, or may comprise portions of steps.

The heart splints as disclosed herein may be utilized in combination with heart valve prosthetics, heart valve repair implants, or other devices, systems, or apparatuses disclosed herein may be utilized as desired. For example, such heart splints may be utilized in combination with an annuloplasty ring, or other annuloplasty devices, or other form of device for repair of a heart valve annulus.

The “user” as discussed herein may comprise a user of the systems and apparatuses disclosed herein, which may include a surgeon, or another individual such as a medical professional who may operate the systems and apparatuses disclosed herein, without limitation.

The present disclosure offers numerous advantages over existing treatments for various heart conditions, including valve incompetencies. The devices disclosed herein do not require the highly invasive procedures of current surgical techniques. For instance, the treatments described herein do not require removing portions of heart tissue, nor do they necessarily require opening the heart chamber or stopping the heart during operation. The methods of the present disclosure may comprise beating-heart repair of or treatment of the patient's heart. For these reasons, the treatments and techniques for implanting the devices of the present disclosure convey a reduced risk to the patient as compared with other techniques. The less invasive nature of the treatments and techniques and tools of the present disclosure may further allow for earlier intervention in patients with heart failure and/or valve incompetencies. While often discussed herein in terms of mitral valve treatments, the systems, devices, methods, etc. may be used to treat other heart valves, heart conditions, enlargement of other organs, etc.

Although the present disclosure is discussed in connection with treating the mitral valve and tricuspid valve of the heart, the present disclosure may be applied to various chambers of the heart and for other valves of the heart for similar purposes. More broadly, the systems, apparatuses, methods, etc. disclosed herein may be used in other applications to change the geometries and/or stresses of other parts of the body (e.g., a stomach, bladder, or other part of the body).

The apparatuses and other devices disclosed herein may be practiced separately as desired. In addition, the methods herein are not limited to the methods specifically described, and may include methods of utilizing the systems, apparatuses, and devices disclosed herein.

In closing, it is to be understood that although aspects of the present specification are highlighted by referring to specific examples, one skilled in the art will readily appreciate that these disclosed examples are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular examples only, and is not intended to limit the scope of systems, apparatuses, and methods as disclosed herein, which is defined solely by the claims. Accordingly, the systems, apparatuses, and methods are not limited to that precisely as shown and described.

Certain examples of systems, apparatuses, and methods are described herein, including the best mode known to the inventors for carrying out the same. Of course, variations on these described examples will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described examples in all possible variations thereof is encompassed by the systems, apparatuses, and methods unless otherwise indicated herein or otherwise clearly contradicted by context.

Groupings of alternative examples, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method for approximating papillary muscles of a heart, the method comprising:
deploying a first heart anchor to an external posterior surface of the heart proximate a papillary muscle of the heart;
deploying a second heart anchor to an external anterior surface of the heart proximate a papillary muscle of the heart;
tensioning a tension member for coupling the first heart anchor to the second heart anchor; and
locking the tension member in tension between the first heart anchor and the second heart anchor across a ventricle of the heart.

2. The method of claim 1, wherein the second heart anchor is deployed to the external anterior surface of a left ventricle.

3. The method of claim 1, wherein the first heart anchor is deployed to the external posterior surface of a left ventricle.

4. The method of claim 1, wherein the first heart anchor or the second heart anchor includes:
a ring having two ends and configured to move from a linearized configuration to a ring-shaped configuration; and
a cover coupled to the ring and extending inward from the ring in the ring-shaped configuration.

5. The method of claim 4, wherein the ring is configured to be in the linearized configuration in an unexpanded configuration and configured to be in the ring-shaped configuration in an expanded configuration.

6. The method of claim 1, wherein both the first heart anchor and the second heart anchor include:
a ring having two ends and configured to move from a linearized configuration to a ring-shaped configuration; and
a cover coupled to the ring of a respective one of the first heart anchor or the second heart anchor and extending inward from the ring of the respective one of the first heart anchor or the second heart anchor in the ring-shaped configuration.

7. The method of claim 1, wherein the first heart anchor includes a pad and the second heart anchor includes a pad and a lock for locking the tension member to the second heart anchor.

8. The method of claim 1, wherein the second heart anchor includes a ratchet mechanism configured to allow the tension member to be drawn through the second heart anchor in a first direction and resist movement of the tension member in a second direction that is opposite the first direction.

9. The method of claim 1, further comprising moving the first heart anchor from an unexpanded configuration to an expanded configuration at the external posterior surface of the heart.

10. The method of claim 1, further comprising deploying the first heart anchor to the external posterior surface of the heart, the first heart anchor configured to have a static size.

11. The method of claim 1, further comprising:
passing a deployment apparatus through the external posterior surface of the heart, the deployment apparatus including an implant retention area for retaining the first heart anchor in an unexpanded configuration; and
deploying the first heart anchor to the external posterior surface of the heart from the implant retention area.

12. The method of claim 11, further comprising:
passing an elongate shaft of the deployment apparatus from an atrium into the ventricle; and
steering a steerable distal end of the elongate shaft to position a puncturing tip of the elongate shaft to pass through the external posterior surface to deploy the first heart anchor at the external posterior surface and to position the puncturing tip to pass through the external anterior surface to deploy the second heart anchor at the external anterior surface.

13. The method of claim 12, further comprising:
puncturing the external anterior surface of the heart with the puncturing tip of the deployment apparatus; and
extending the deployment apparatus across an interior cavity of the ventricle from the external anterior surface to the external posterior surface.

14. The method of claim 1, further comprising:
puncturing the external anterior surface with a needle, the needle having an interior lumen;
puncturing the external posterior surface with the needle;
extending the needle across an interior cavity of the ventricle from the external anterior surface to the external posterior surface; and
deploying a snare from the interior lumen of the needle external of the external posterior surface.

15. The method of claim 14, further comprising utilizing the snare to extend the tension member across the interior cavity of the ventricle.

16. The method of claim 1, further comprising:
drawing the first heart anchor to the external posterior surface of the heart; and
coupling a proximal portion of the tension member to the second heart anchor.

17. The method of claim 1, further comprising:
extending the tension member from the first heart anchor across an interior cavity of the ventricle to the external anterior surface; and
coupling a proximal portion of the tension member to the second heart anchor.

18. The method of claim 1, further comprising tensioning the tension member during a beating heart procedure.

19. The method of claim 18, further comprising rapid pacing the heart.

20. The method of claim 1, further comprising:
puncturing a posterior medial papillary muscle of the ventricle;
puncturing an anterior lateral papillary muscle of the ventricle; and
extending the tension member across the ventricle through the posterior medial papillary muscle and through the anterior lateral papillary muscle,
wherein the first heart anchor is deployed to the external posterior surface of the heart proximate the posterior medial papillary muscle, and
wherein the second heart anchor is deployed to the external anterior surface of the heart proximate the anterior lateral papillary muscle.
